(19) United States
(12) Patent Application Publication
(10) Pub. No.: US 2019/0344015 A1
(43) Pub. Date: Nov. 14, 2019

(54) Secure Bolus Control System

(71) Applicant: Debiotech S.A., Lausanne (CH)

(72) Inventors: Frédéric Neftel, Crans-Montana (CH); Stephan Proennecke, Lausanne (CH); Arnaud BELLADON, Collonges-sous-Salève (FR)

(21) Appl. No.: 16/511,011

(22) Filed: Jul. 15, 2019

Related U.S. Application Data
(62) Division of application No. 15/508,917, filed on Mar. 4, 2017.

(30) Foreign Application Priority Data
Oct. 17, 2014 (EP) 14189454.3

(51) Int. Cl. A61M 5/172; A61M 5/142

(52) U.S. Cl. CPC A61M 5/172; A61M 2205/3334; A61M 2205/276; A61M 2205/3584; A61M 2205/50; A61M 2205/502; A61M 2205/609; A61M 2205/8206; A61M 2005/14208; A61M 2005/14268; A61M 2205/6072; A61M 2205/6054; A61M 2205/6018; A61M 2205/582; A61M 2205/581; A61M 2205/52; A61M 2205/505; A61M 2205/3569; A61M 2205/3317; A61M 2205/3306; A61M 2205/587; A61M 5/14248

(57) ABSTRACT

A delivery device comprising an acquisition mode for activating at least one button for controlling the delivery of a drug.

### FIELD OF THE INVENTION

The invention relates to the general field of devices for delivering a medical fluid to a patient, particularly for commanding a bolus using input means (for example a button) arranged on the delivery device. The present application claims the priority of EP 14189454.3 filed on Oct. 17, 2014 in the name of Debiotech, the integral content of which is to be considered to form part of the present application.

### PRIOR ART

Certain therapies may or have to be applied to the patient using a delivery device that allows a solution (insulin, analgesic, etc.) to be administered to a patient. These devices generally comprise a pump, a processor, and control means. These control means may allow the patient or the care personnel to program a delivery in advance or command same at an instant t. There are two types of delivery:
the basal which is a delivery of the solution continuously, or at least over a relatively long period, at a programmed rate that may evolve over time,
the bolus which is a temporary delivery of a given quantity, over a relatively short or determined time.

It is well known to those skilled in the art that the basal delivers the solution at a lower flow rate than the bolus and over a longer period.

Delivery systems have been undergoing constant development for over twenty years. The first systems comprised all the elements necessary for programming and delivery in one single device. Thus, the two elements were physically inseparable for the operation of the system. For example, this type of device was divulged in American patent U.S. Pat. No. 4,498,843 B2, the entire content of which is incorporated by reference into the present document. These devices thus comprised all the programming buttons, a screen, a reservoir and a pumping mechanism. The patient could attach the device to his belt, and he had then to remove it from his belt in order to program it so as to be able to look at the programming screen. These devices also comprised a flexible tube leading from the device to the cannula (used to infuse the solution into the patient's body). These devices were bulky, heavy and somewhat impractical. In addition, despite the incorporation of a screen, the latter was often small and did not allow easy and safe programming.

Another generation of delivery system allowed the programming means to be dissociated from the delivery means. Each of the two means is arranged in one of two distinct devices, operating physically separately from one another. However, it is essential to have both devices in order to be able to control or monitor the delivery system. Such a system was divulged in international application WO 01/76684 A1, the entire content of which is incorporated by reference into the present document. Thus, the patient was able to have a remote control allowing him to program his delivery device. However, whenever this remote control developed a fault or was mislaid, the patient was unable either to command a delivery or to monitor his system. In the case of an insulin delivery system, the consequences may be dangerous to the health of the patient because after a meal, if the patient cannot command a bolus of insulin, he will then become hypoglycemic.

The latest generations of delivery system comprise a remote control that allows the control and monitoring of the delivery device which itself comprises input buttons allowing only the command of a preprogrammed bolus. Thus, this system allows the patient to command the delivery of a solution either via the remote control or directly via his delivery device. Unfortunately, the delivery device does not allow as much flexibility in the programming as can be achieved with the remote control because the input button does not allow the quantity to be varied and has no use other than that of initiating the delivery. In addition, this new generation is not without risk because it is necessary to take care to ensure that the buttons of the delivery device are not activated or actuated unintentionally.

Specifically, in some instances, the solution delivered may be particularly dangerous to the patient. It is thus important to make sure that each delivery command is necessary to the therapy. In other words, it is important that a command should not be issued unintentionally, because this command could be dangerous to the patient. For example, in the case of insulin, too great a delivery of insulin may induce a hypoglycemic coma or even cause the death of the patient.

As described hereinabove, these new delivery devices comprise input buttons (generally referred to as bolus buttons) to command the delivery of a bolus. Now, these buttons can be actuated unintentionally by the patient or by any other object coming into contact with these buttons. The patient may therefore, without wishing to do so, receive a lethal dose of the solution. In order to guard against such risks, certain delivery devices are equipped with several buttons and with a security mechanism. A device of such a type is described in international application WO 2009/013736 A1, the entire content of which is incorporated by reference into the present document. In that application, the invention set out allows the patient to trigger the pumping mechanism to deliver a preprogrammed bolus of the solution simply by actuating the two buttons of the delivery device. The security mechanism checks the actuation of the buttons and prevents the delivery signal from being sent if the two buttons are not actuated according to a particular sequence or at the same time.

According to that document, such a device would make it possible to improve patient safety. However, the solutions proposed by that document are not optimal. Specifically, they force the patient to remember a determined sequence that sets off the delivery. The more complex this sequence the better the security because the probability of performing this sequence unintentionally becomes lower. In the case of insulin, an unintentional injection could cause the death of the patient, and so it is of key importance that this sequence be complex. The patient therefore has to learn this complex sequence, something which does not come readily to everybody. In addition, if this sequence can be customized to the patient, it is certain that, in order to make use easier the patient will program a sequence that is easier to remember and therefore not complex enough. Now, that considerably increases the probability of this sequence being input unintentionally. Furthermore, the fact that these buttons trigger the delivery directly is dangerous. Specifically, if the preprogrammed bolus is equal to 6 U, then in the event of an unintentional actuation, the entirety of these 6 U will be delivered, and this once again increases the dangerousness of this system.

A mechanical/physical solution (a cap, a protective element, etc.) is possible but this is also restrictive. For example, caps may become detached or make the buttons difficult or even impossible to access when the delivery device is being worn under clothing.

In order to increase safety what is needed is a means or a method that only the user (patient, care personnel, etc.) is capable of performing and the device needs to be capable of recognizing the user's intention. Furthermore, this means or method must not represent a major difficulty of manipulation because otherwise the user will not use this functionality or will reduce the level of security. The issue is therefore one of giving the device greater intelligence, ensuring greater security against unintentional commands while at the same time conforming to a certain simplicity and intuitiveness in the operations required.

### GENERAL DESCRIPTION OF THE INVENTION

According to a first aspect of the invention, one of the objects is to mitigate the disadvantages of the known systems by proposing a security system that prevents any delivery command that is not truly intentional.

According to the invention, the system comprises a delivery device (for example a patch pump) and a means of activating an acquisition mode. The delivery device further comprises at least one button which may be designed to allow the commanding of a certain quantity of solution to be administered to the patient when the delivery device is in acquisition mode. Thus, for preference, as long as the delivery device is not in acquisition mode, said at least one button does not allow the programming of a delivery command.

The acquisition mode may furthermore allow the opening of a time window during which the patient may command the administration a certain quantity of solution using said at least one button. The means of activating this mode and the acquisition mode itself allow security to be applied to the device and the use of the delivery command means (said at least one button).

For preference, the delivery device may comprise audible, visual and/or tactile indicators designed to inform the patient of:
the activation of the acquisition mode, and/or
the opening and/or the closing of the time window, and/or
the commanded and recorded quantity that will be administered to the patient, and/or
the impossibility of delivering the commanded dose, and/or
the canceling of the command, and/or
the actual delivery of the required dose.

According to one embodiment, the activation means is separate from said at least one button. For preference, the activation means does not use said at least one button. Furthermore, the activation means may be separate from the delivery device.

For preference, the activation means is an element that the patient always retains in his possession, such as a token, an element distinct from but indispensable to the delivery device (a patch, a cannula), a mobile phone, a magnet or a keyring.

According to one aspect of the invention, the present document divulges a method for commanding the administration of a medical fluid to a patient, which comprises the following steps:
providing a portable medical device designed to be fixed/attached to the body of the patient, the medical device comprising:
   a reservoir containing the medical fluid to be administered to the patient,
   a processor,
   a pumping mechanism controlled by the processor, designed to administer the fluid contained in the reservoir to the patient,
   a sensor designed to send a signal to the processor,
   a button designed to send a signal to the processor, and
providing an activation device designed to activate the sensor,
activating the sensor a first time so that the medical device initiates an acquisition mode allowing the programming of a given quantity of medical fluid to be administered,
activating (or actuating or pressing) the button at least once so as to program a given quantity of fluid to be delivered,
optionally, activating the sensor a further time so as to send the signal to the processor to deliver the fluid according to the given quantity that has been or will be programmed using the button.

This method may make it possible to program a bolus without the aid of a programming screen, but this does not mean that the system has no screen. However, patch pumps generally have no screen, the screen being carried remotely on the remote control.

The present document divulges other inventions that may use a delivery system similar to the one divulged hereinabove. These other inventions afford even greater security for the patient and his therapy.

According to one aspect of the invention, the present document divulges a method for commanding the administration of a medical fluid to a patient which comprises the following steps:
providing a medical device comprising:
   a wireless communication module designed to receive and/or send data from and/or to a distant remote control,
   at least one button, and
   a processor electronically connected to the wireless communication module and to the button,
the processor receiving a signal following activation of at least one button,
determining whether the wireless communication module of the medical device is able to exchange data with the distant remote control,
initiating the delivery of the medical fluid if the wireless communication module of the medical device is unable to exchange data with the distant remote control.

By virtue of this system or method, the patient can command an administration using the control button of the medical device (which is preferably the delivery device) if, and only if, the medical device is unable to exchange data with the distant remote control. For example: if the remote control has no battery left, if the remote control is not correctly paired with the medical device, if the remote control is lost or broken, if the remote control is not in communication range (radiofrequency, WiFi, Bluetooth, IR, etc. range) with the medical device or if one of the communication devices is defective, etc. Thus, the patient will be encouraged to use his remote control when the latter can be used, because if the patient wishes to program a delivery he will then need to do so using his remote control.

According to one embodiment, the medical system may comprise:
a delivery device comprising:
   a reservoir containing the medical fluid to be administered to the patient,
   a processor,
   a pumping mechanism controlled by the processor, designed to administer the fluid contained in the reservoir to the patient, and
   a button designed to send a signal to the processor,
a remote control distant from the delivery device and designed to control and/or monitor the delivery device, comprising:
   a processor, and
   an audible or visual indicator connected to the processor.

When the button of the delivery device is activated, the audible or visual indicator is activated by the processor of the remote control.

By virtue of this system, the patient is encouraged to favor programming via the remote control over programming via the control button of the device. In addition, this functionality allows the user easily to find his remote control if it has become misplaced.

According to one embodiment, the delivery device may comprise:
a reservoir containing the medical fluid to be administered to the patient,
a processor,
a pumping mechanism controlled by the processor, designed to administer the fluid contained in the reservoir to the patient,
an audible or tactile indicator,
a button designed to send a signal to the processor, and
a memory coupled to the processor designed to record the data of an administration command.

Once the button has been activated (by the user by pressing the button), the audible or tactile indicator transmits to the patient in pulses at least one data item relating to the previous administration or a data item about the current administration or any other data known to the delivery device. The pulses may be audible, luminous and/or tactile, in which case the indicator may be a mini speaker or a vibrator or a light source such as an LED. Thus, even without a screen or without a remote control, the patient may determine the quantity delivered in the previous command, the quantity in the process of being delivered, the current flowrate (of the basal or of the bolus), the time of the previous delivery, the quantity of drug currently present in the patient's body but not yet used (for example the IOB which may be calculated by the processor as a function of the administration data), the level in the reservoir, etc.

According to one aspect of the invention, the present document divulges a method which comprises the following steps:
providing a medical device comprising:
   a reservoir containing the medical fluid to be administered to the patient,
   at least one input button,
   a processor electronically connected to the input button,
   a pumping mechanism controlled by the processor, designed to administer the fluid contained in the reservoir to the patient, and
   a memory designed to store therapy data, for example data relating to the quantity of fluid previously administered and/or to the maximum quantity of fluid that can be administered to the patient,
activating the input button so as to program an administration command
sending the processor therapy data, for example the data relating to the quantity of fluid previously administered and/or to the maximum quantity of fluid that can be administered to the patient,
using the processor to limit the administration command as a function of the therapy data, for example the data relating to the quantity of fluid previously administered and/or to the maximum quantity of fluid that can be administered to the patient.

By virtue of this method, the system ensures that the patient cannot command a dose of medical fluid that will be above a threshold determined by the therapy at the moment at which he programs his command. In particular, the processor may, using a mathematical model, calculate the maximum admissible dose for the patient when the latter is proceeding to program the command, which may be a function of the quantity of fluid previously administered and the maximum quantity that the patient can receive. In other words, if the fluid is insulin, the processor may calculate the quantity of insulin previously administered and from this deduce the quantity of insulin injected into the patient's body but not yet used at the moment at which he is programming the command. The processor may search the memory of the device for the maximum quantity that can be administered, which may be a preprogrammed data item dependent for example on the therapy recommended by the doctor. Thus, the quantity that the patient will be able to command using the button will be less than or equal to the maximum quantity that can be administered minus the IOB. If the patient commands a greater quantity, then the medical device will inform the patient that it cannot deliver more than a certain quantity (via an audible or tactile or luminous indicator).

### LIST OF FIGURES

The invention will be better understood hereinafter by means of a number of illustrated examples. It goes without saying that the invention is not restricted to these embodiments.

FIG. 1 illustrates an embodiment using a token.

FIG. 2 sets out a view in cross section of one embodiment the device of which is connected to a patch.

FIG. 3 divulges one possible embodiment comprising two buttons.

FIG. 4 illustrates a delivery command method.

FIG. 5 sets out an exploded view of a delivery device.

FIG. 6 sets out an illustration of a delivery system using a remote control and a separate delivery device.

FIGS. 7a, b and c show possible displays on the remote control.

FIGS. 8a and b illustrate one possible method for programming a bolus.

FIG. 9 sets out a 3-D and cross-sectional view of a patch that the invention may use.

FIG. 10 illustrates possible steps in a method of programming a bolus.

FIG. 11 illustrates possible steps in a method of programming a bolus.

FIG. 12 illustrates possible steps in a method of programming a bolus.

FIG. 13 illustrates possible steps after a method of programming a bolus.

FIG. 14 illustrates possible steps after a method of programming a bolus.

FIG. 15 illustrates possible steps in a method of programming a bolus.

FIG. 16 sets out a screen for customizing the control buttons.

### NUMERICAL REFERENCES USED IN THE FIGURES

1 Delivery device
2 Control button or bolus button
3 Token
4 Action of bringing the token closer to the device
5 Patch
6 Patient's skin
7 Adhesive
8 Cannula
9 Hall effect sensor
10 Magnet
11 Optional shell protecting the control button or buttons
100 Delivery device
200 Remote control
201 Screen
202 Button
203 Communication
204 Control button
300 Main screen
301 Information banner bearing information regarding the last bolus infused
302 Bolus infusion progress display

### DETAILED DESCRIPTION OF THE INVENTION

In the present document, the detailed description of the invention comprises embodiments of devices, of systems and of methods which are given by way of illustration. Of course other embodiments are conceivable and may be applied without departing from the scope or spirit of the invention. The detailed description which follows therefore must not be considered as being limiting.

Unless indicated otherwise, the scientific and technical terms used in the present document have the meanings commonly employed by those skilled in the art. The definitions given in this document are given to assist with understanding the terms frequently used and are not intended to limit the scope of the invention.

The direction indications used in the description and the claims, such as “top”, “bottom”, “left”, “right”, “upper”, “lower”, and other directions or orientations are mentioned in order to provide greater clarity with reference to the figures. These indications are not intended to limit the scope of the invention.

Verbs like “have”, “comprise”, “include” or equivalent are used in the present document in a broad sense to mean, in general, “includes, but is not restricted thereto”.

The term “or” is generally used in a broad sense encompassing “and/or” unless the context clearly indicates the contrary.

Description of the Delivery Device and of These Optional Accessories

The inventions described in this document may use a delivery device as set out in FIG. 5. That figure divulges a delivery device (100) that may comprise a reservoir (103) containing a medical fluid to be administered to a patient, electronic elements (106) which may be powered by a battery (109), at least one casing (104, 107), a sensor (108), a pumping mechanism (not visible in FIG. 5) designed to administer the fluid contained in the reservoir to the patient, and a button (not visible in FIG. 5). The pumping mechanism may be a syringe driver system, a membrane pump or a pressurizing system. For preference, the pumping mechanism is a micropump because it allows the delivery device to be extensively miniaturized; such a micropump is divulged in international patent application WO 01/90577 A1, the entire content of which is incorporated by reference into the present document.

The delivery device may comprise a disposable part (101) and a reusable part (102), the reusable part potentially being used in succession with several disposable parts. The reusable part may comprise the more expensive and/or complex elements such as, for example, certain electronic elements, one or more buttons, one or more sensors (108). The electronic elements may comprise at least one processor and a memory arranged on a printed circuit. The sensor (108) and at least one button may be connected to the processor and they may be connected to a printed circuit preferably connected to the processor. It goes without saying that the sensor is an element distinct and different from the button (for example structurally or functionally different) and that it cannot be activated in the same way. The button may be activated by actuating the latter by, for example, pressing it. In that case, the sensor is activated by a stimulus that is not the result of pressing it, for example the sensor may be activated by an activation device. The sensor may send signals, for example measurement signals or signals upon detection of an element (for example detection of the magnetic field of a magnet or of a bar code or of an identification code sent by RFID, etc.). A similar device is divulged in international application WO 2007/113708 A1, the entire content of which is incorporated by reference into the present document.

For preference, the processor and the memory are arranged in the reusable part (102). The sensor may also be arranged in the reusable part (102). The sensor may further be arranged in the casing (107) of the reusable device. The button for programming a bolus may be arranged on the casing (107) of the reusable device.

The button and the sensor are designed to send signals to the processor. The processor is capable of distinguishing the signals coming from the button from those coming from the sensor. It is also capable of initiating actions that differ according to the signal and/or according to the situation (remote control within communication range, etc.).

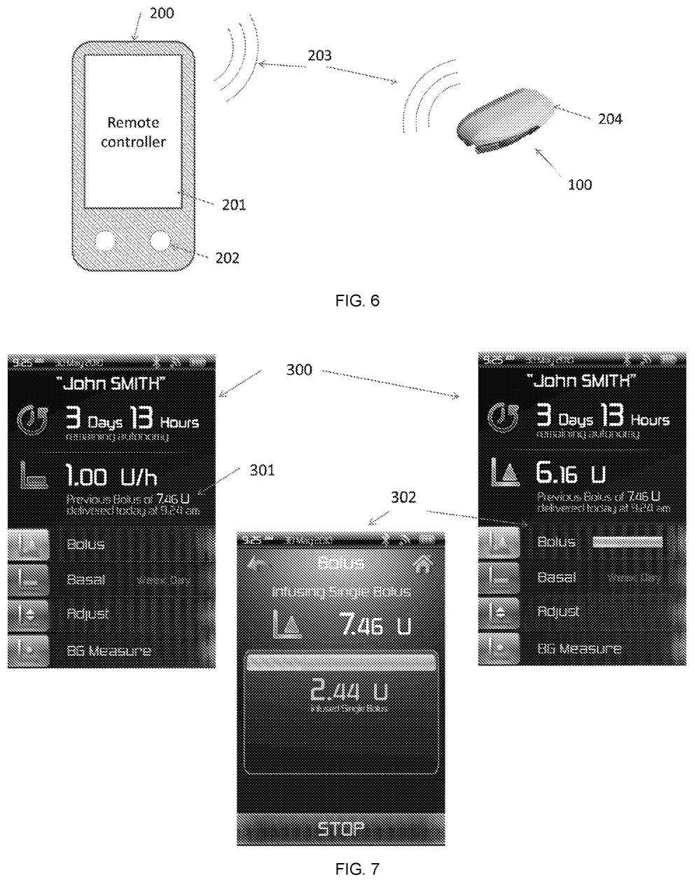

The inventions described in this document may use a medical system as set out in FIG. 6. This medical system divulged may comprise a delivery device like the one described hereinabove and a remote control designed to control, monitor and/or program the delivery device. A similar system is divulged in international application WO 2014/009876 A1, the entire content of which is incorporated by reference into the present document. This remote control may comprise a communication module designed to send and/or to receive (203) data of the delivery device which may also comprise a communication module. The communication module comprises all the elements necessary for its operation (antenna, etc.) as known by those skilled in the art. The communication devices may be designed to use a communication protocol such as Bluetooth, WiFi, etc.

The remote control (200) may comprise a screen (201), at least one button (202) and a processor connected to these elements and to the communication module. The screen may be a touch screen, it may be designed to display at least one screen divulged in FIGS. 7a, b and c but also at least one programming screen to program the administration of the medical fluid (basal and/or bolus). The screen may be designed to display information coming from the delivery device, such as the previous administration or the administration in progress that would have been commanded via the control buttons of the delivery device. These data may be displayed directly on the home screen of the remote control. The screen may be designed to display a succession of displays, the first of which may be the display of the home screen or basic screen.

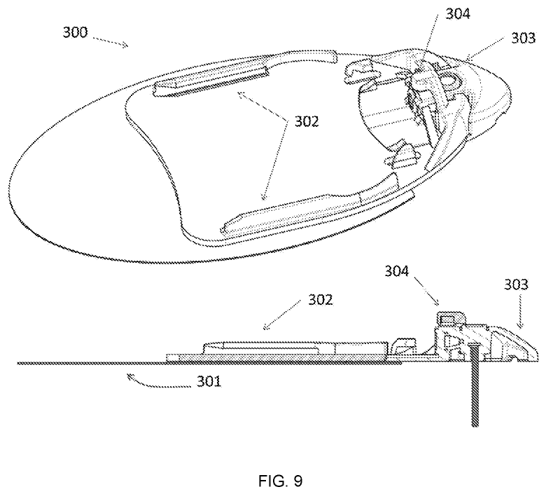

The medical system may further comprise a patch designed to fix the delivery device against the patient's body, preferably to the patient's skin. Such a patch is divulged in international application WO 2013/068900 A1, the entire content of which is incorporated by reference into the present document. FIG. 9 divulges a 3-D view and a view in cross section of a patch (300) that may comprise an adhesive lower surface (301) designed to be brought into contact with the patient's skin and mechanical coupling elements (302) designed to fix the delivery device removably to the patch. The patch may also comprise a transcutaneous device (303) which comprises a fluidic path extending underneath the lower surface of the patch (for example a micro-needle, a flexible or rigid cannula). The transcutaneous device (303) allows the delivery device to inject and/or to infuse the solution directly into the patient's body. Thus, the delivery device may be a patch pump designed to be attached directly or indirectly to the patient's skin and preferably comprises no flexible tube (that would be accessible while the system is in operation) between the pump and the transcutaneous device. According to one embodiment, the transcutaneous device can be detached from the patch.

The transcutaneous device (303) allows fluidic communication between the delivery device and the patient's body (preferably) when the delivery device is fixed/attached to the patch. In other words, delivery of the solution to the patient's body can be performed only if the delivery device is coupled, arranged on or against or connected to said patch or at least to the transcutaneous device (303).

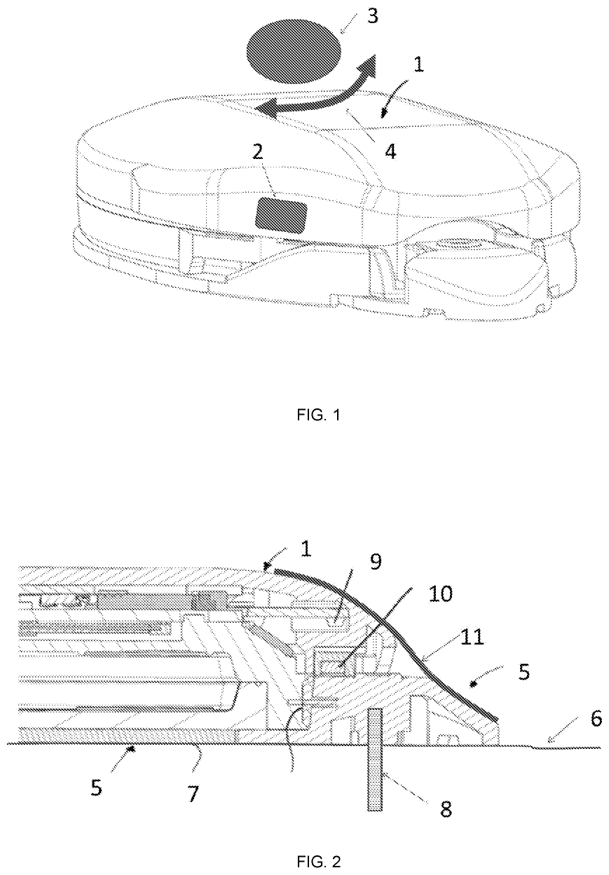

The patch and/or the transcutaneous device may comprise a mechanical, visual, electromagnetic or electronic element making it possible for the device to determine whether it is actually coupled or connected to said patch and/or to said transcutaneous device. This element may activate a sensor of the delivery device so that the latter sends a signal to the processor. This may be a magnet fixed or arranged in or on the body of the patch or of the transcutaneous device. A view in cross section of the entire delivery device connected to patch assembly is divulged in FIG. 2. In that figure, the delivery device (1) is coupled (or connected) to a patch (5) stuck (via an adhesive (7) for example) to the skin (6) of a patient. The patch (5) may comprise a cannula (8). The delivery device may comprise a Hall effect sensor (9) connected to a processor (not depicted) designed to collaborate with a magnet (10) of the patch.

In one embodiment, a simple and economical solution that would allow the button or buttons of the delivery device not to be actuated might be a shell (11) covering the button or buttons when the delivery device is connected or coupled to the patch. Thus, when the delivery device is removed from its patch, the control button or buttons become accessible and can be used by the patient to program a quantity to be delivered. This shell may be an element of the patch and/or of the transcutaneous device that extends over the button or buttons, rendering them inaccessible. Thus, when the delivery device is installed on the patch, the device passes partially under this shell so as to protect the control button or buttons.

Possible Programming Methods

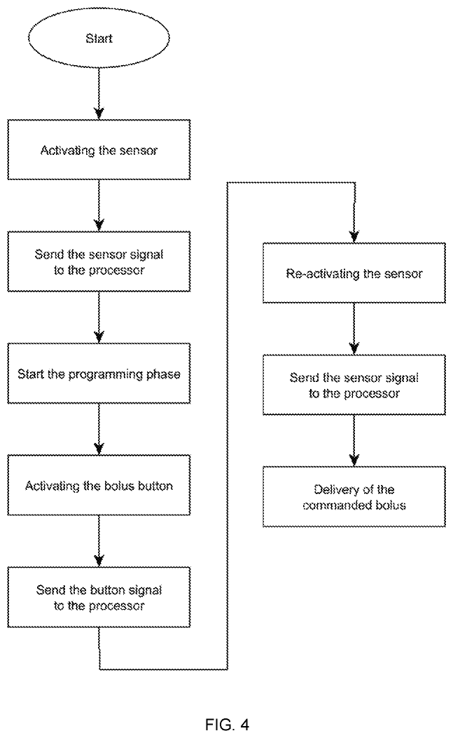

For preference, the delivery device comprises at least one button (referred to as an input button, programming button or bolus button) allowing the command of a quantity of solution to be delivered to the patient and an activation means allowing activation of a mode referred to as “acquisition mode”. This activation means may also be referred to as activation device or additional device. It may be separate from the delivery device and/or may be indispensable to the latter for its normal operation (allowing the delivery of the medical fluid to the patient). This activation means is designed to interact with a sensor of the delivery device. In one embodiment, the acquisition mode is initiated by activation of the sensor via the activation means, but for greater security the patient needs to apply a long pressure to the bolus button (within a certain lapse of time) to confirm or activate acquisition mode. Thus, the delivery device is able to detect the intention of the user to command a bolus via the buttons of the delivery device. When the patient wishes to program a bolus directly via the delivery device, the patient uses this activation means to activate the acquisition mode. A first example of a method of programming a bolus as divulged by the invention is illustrated in FIG. 4.

This activation means may be a token used as third-party security. When this token is brought close to the delivery device (optionally the patient also actuates a control button), said device interprets this action (or this succession of actions) as an intention on the part of the user to use the control button or buttons. Thus, the token (or the token and the actuation of the button) allow the opening of a time window (activation of an acquisition mode) during which the user can use said at least one button to command a certain quantity of solution for the patient.

The time window corresponds to a given duration for which the user can use the button or buttons to command a delivery (preferably a bolus). This time window is opened following activation of the acquisition mode. The delivery device may comprise an audible or visual or tactile indicator to inform the patient when this window is open and/or whether it is still open and/or when it closes.

As long as this time window is open, the patient can press the bolus button or buttons to determine the quantity of solution to be administered. The duration of this time window may be fixed or lengthened for each actuation of the bolus button or buttons. In other words, there are two solutions:
either the duration is defined and the user must, during this lapse of time, command his delivery, for example the duration may be set between 10 seconds and 5 minutes;
or the duration is variable and dependent on the last use of the button or buttons for example, the time window closes if the user has not pressed the bolus button or buttons for a period fixed at between 5 seconds and 5 minutes.

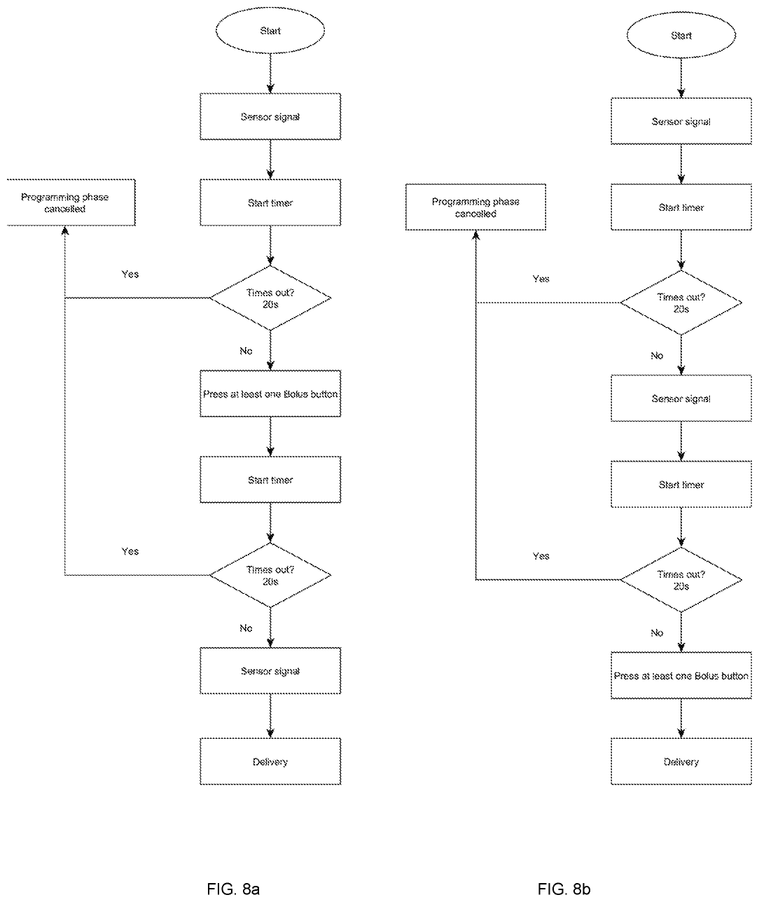

FIGS. 8a and 8b set out two possible programming methods. FIG. 8a divulges a method using the following successive steps:
first activation of the sensor,
actuation of the control button,
further activation of the sensor,
delivery of the commanded quantity.

FIG. 8b divulges another possible method involving the following successive steps:
first activation of the sensor,
further activation of the sensor,
actuation of the control button,
delivery of the commanded quantity.

In the method of FIG. 8b, the first activation of the sensor is followed directly by the further activation of the sensor. Thus, in order to program the quantity of fluid to be delivered, the control button can be actuated after the further activation of the sensor. Optionally, the sensor can be activated once again after activation of the control button so as to confirm the command before the command is executed. Thus, for example, the token can also be used to confirm the command.

In both instances, the delivery device may be already fitted to the patient's body. The activation means will be used to activate the sensor for a first time and the sensor will then send a signal to the processor. Following this first activation, the processor starts a countdown during which the patient needs either to activate the sensor again or to activate/actuate the control button of the delivery device at least once. Following this step, a new countdown may be started and must be followed by a further action. If no action is performed during one of the countdowns then the programming may be canceled (the time window is then closed again). The delivery device may ring or vibrate to indicate that the programming has begun and/or that the programming has been canceled.

The token may be:
an RFID chip,
a bar code that the device is capable of reading and recognizing,
a mechanical component or an electronic device designed to be coupled temporarily to the delivery device (before, during or after actuation of the buttons),
an NFC (near field communication) system, for example an NFC tag that can be used via a mobile phone or any other electronic device,
an electronic signature,
a fingerprint, in which case the device comprises the electronic elements needed for reading and recognizing a fingerprint,
a magnet which is recognized by a detector (for example a Hall effect detector) contained in the device.

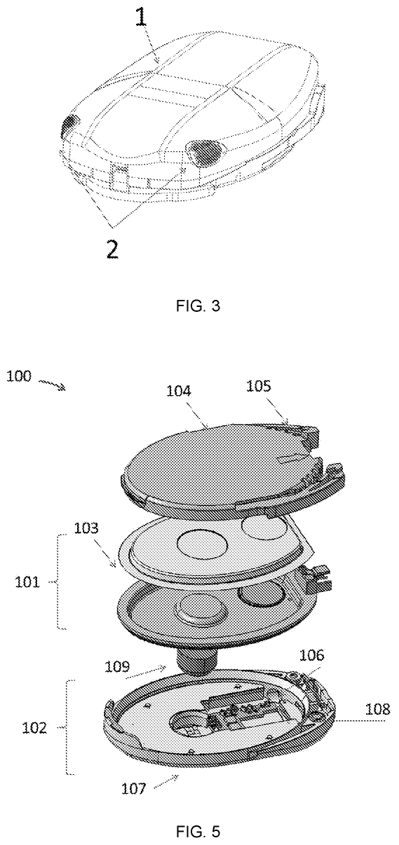

FIG. 1 sets out an example of a delivery device (1) which comprises at least one button (2) and an activation means (3) here depicted as a token which is passed in the direction of the arrow (4) over the delivery device (1). The delivery device may comprise two buttons (2) positioned one on each side of the device, as divulged in FIG. 3.

For preference, in order to avoid the addition of an expensive sensor designed for this functionality alone, the activation means interacts with a sensor of the delivery device which is also designed for some other function, for example the sensor may be designed to send the processor a signal when the portable medical device is removed from and/or fitted to the patient's body. Thus, to program a bolus, the patient may simply remove and refit the delivery device from and onto his body.

In particular, the removal of the delivery device from its patch (or from the transcutaneous device) will be detected by the sensor which will send a signal to the processor. The same is true when the delivery device is refitted to the patient's body. The patient therefore has a certain length of time in which to actuate the control buttons to initiate programming of the quantity to be delivered. This manipulation cannot be performed unintentionally, and is simple to remember and to perform.

This sensor may be a Hall effect sensor which cooperates with the magnet of the patch (or of the transcutaneous device). Upon each connection and disconnection, the Hall effect sensor will be activated by the magnet of the patch (or of the transcutaneous device) and will send a signal to the processor. If necessary, a token as described hereinabove may also be used. For example, another magnet may be used to activate the sensor without having to disconnect/reconnect the delivery device. What happens is that the Hall effect sensor will detect the passage of the token (magnet) and the processor will open a time window for programming a bolus using the buttons. This new magnet may be designed to temporarily (as it passes over same) neutralize the effects of the magnet in the patch on the sensor and thus make the processor believe that the delivery device has been disconnected from the patch. According to another embodiment, the processor may discern activation of the sensor using the magnet of the patch or the magnet of the token (or the magnetic field generated by a device comprising a battery, such as a telephone or the like). The method is therefore possible even if the delivery device is situated in a location that does not allow easy manipulation of the delivery device, for example if the delivery device is worn under clothing.

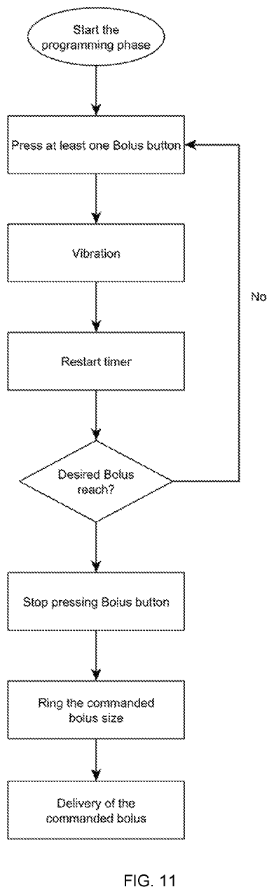

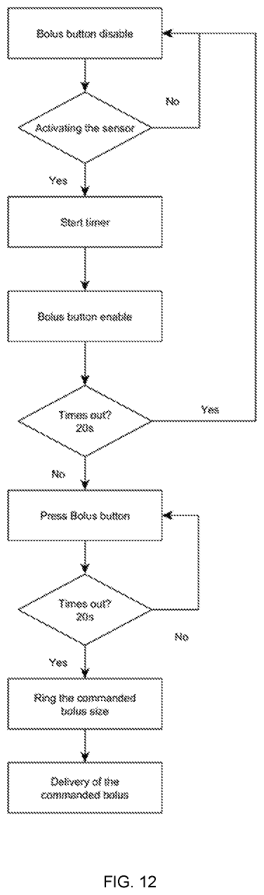

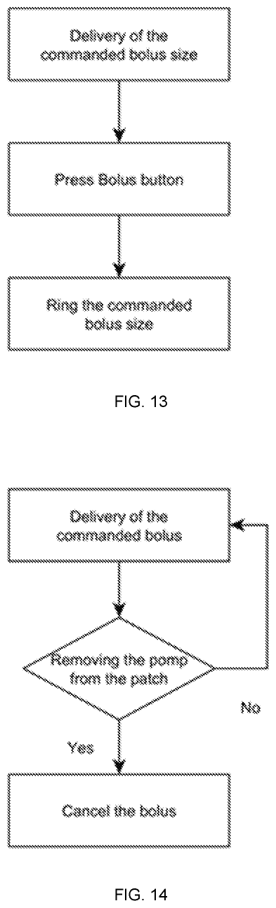

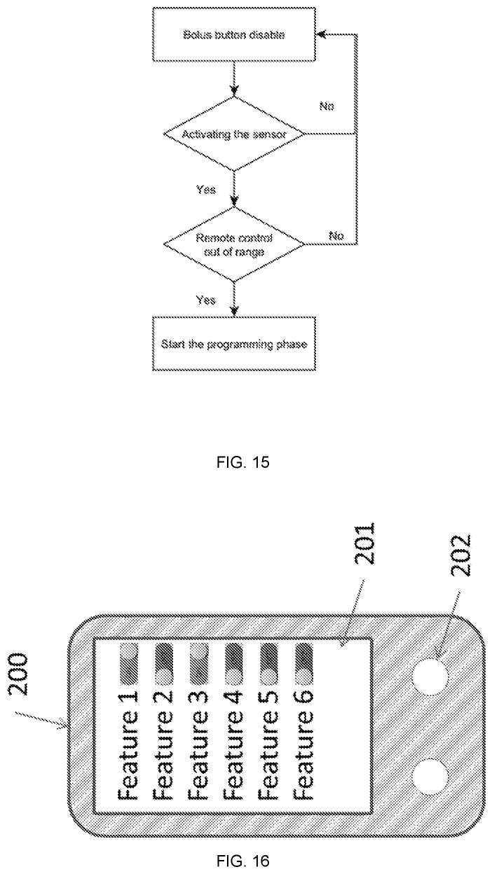

To sum up, according to one possible embodiment, when the patient disconnects or uncouples the delivery device from the patch then the device temporarily enters an acquisition mode which allows the use of the button or buttons to initiate programming of a bolus. For preference, the patient programs the quantity of fluid to be administered by pressing the bolus button once or more than once. FIG. 11 illustrates a bolus programming method that requires one or more actuations of the bolus button or buttons to program the quantity of fluid to be administered. For each actuation of the button, the processor may increment a count which represents the quantity to be infused. For example, if the increment is 0.5 U and the patient wishes to program 3 U then the patient will need to press the bolus button 6 times in succession. Each time the patient actuates the bolus button, a countdown may be started. If, before the end of this countdown, the patient presses again, then the counter will be incremented. If the patient does not press before the end of the countdown then the processor may consider that the patient has finished programming the quantity of fluid to be administered. An audible, tactile or visual (for example a flash of LED) indicator of the delivery device may then inform the patient, for example by pulsing of the indicator, of the programmed quantity, one pulse representing one increment. In our example, the indicator will ring or vibrate or emit light 6 times in succession. Once the quantity has been programmed and provided that the patient is in agreement with this quantity, the patient can reconnect the delivery device to the patch (if this is not already done). The device will then be able to execute the command. To cancel the command, the patient can elect not to reactivate the sensor (for example the patient does not straight away refit the delivery device to the patch), in which case after a certain lapse of time, the command may be canceled.

For preference, as divulged in FIG. 14, if, during execution of the command, the device is once again disconnected or uncoupled, the command may be canceled but the quantity actually administered may be recorded in a memory of the device.

Thus, to command the administration of a medical fluid to a patient, the programming method preferably comprises the following steps:
providing a portable medical device designed to be fixed/attached to the body of the patient, the medical device comprising:
   a reservoir containing the medical fluid to be administered to the patient,
   a processor,
   a pumping mechanism controlled by the processor, designed to administer the fluid contained in the reservoir to the patient,
   a sensor designed to send a signal to the processor,
   a button designed to send a signal to the processor, and
providing an activation device (also known as additional device) designed to activate the sensor,
activating the sensor a first time so that the medical device initiates an acquisition mode allowing the programming of a given quantity of medical fluid to be administered,
activating the button at least once so as to program a given quantity of fluid to be delivered,
optionally, activating the sensor once again so as to send the processor the signal to deliver the fluid in the given quantity that has been or will be programmed using the button.

FIG. 4 illustrates a similar programming method.

The medical device may further comprise a memory to record the data regarding the quantity of fluid commanded. These data may be erased after administration or retained in memory or transmitted to some other device (such as a remote control or a remote server).

According to one possible embodiment, another programming method may comprise the following steps:
providing a portable medical device designed to be fixed/attached to the body of the patient, the medical device comprising:
   a reservoir containing the medical fluid to be administered to the patient,
   a processor,
   a pumping mechanism controlled by the processor, designed to administer the fluid contained in the reservoir to the patient,
   a sensor designed to send a signal to the processor,
   a button designed to send a signal to the processor, and
   a memory coupled to the processor,
providing an additional device, separate from the medical device, designed to activate the sensor,
activating the sensor a first time so that the medical device initiates an acquisition mode allowing the programming of a given quantity of medical fluid to be administered,
activating the button at least once so as to program the given quantity,
recording the given quantity in the memory,
optionally, activating the sensor once again so as to send the processor the signal to deliver the fluid in the given quantity recorded in the memory.

According to one possible embodiment, another programming method may comprise the following steps:
providing a portable medical device designed to be fixed/attached to the body of the patient, the medical device comprising:
   a reservoir containing the medical fluid to be administered to the patient,
   a processor,
   a pumping mechanism controlled by the processor, designed to administer the fluid contained in the reservoir to the patient,
   a sensor designed to send a signal to the processor when the portable medical device is removed from and/or fitted to the body of the patient,
   a button designed to send a signal to the processor, and
   a memory coupled to the processor,
activating the sensor a first time so as to send the processor the signal to initiate an acquisition mode allowing the programming of a given quantity of medical fluid to be administered,
activating the button at least once so as to program the given quantity,
recording the given quantity in the memory,
optionally, activating the sensor again so as to send the processor the signal to deliver the fluid in the given quantity recorded in the memory.

In this embodiment and throughout the other embodiments, the additional device may be a token, a magnet, a patch designed to fix the pump to the body of a patient, or a cannula designed to allow the medical fluid to be infused into the body of the patient. It is a device or a single element designed to be detected or recognized by the sensor. Thus, to activate the sensor, all that is required is for the additional device and the medical device to be at least temporarily brought closer together (or further apart). Activation of the sensor may be the result of the two devices being brought closer together or moved further apart (or both in succession). Activation of the button is preferably the result of this button being pressed by the user. The signals sent may be on, off, an analog signal or a binary signal.

Thus, the additional device may be a device that is indispensable to the medical device (for example the cannula or the patch) but may be considered to be distinct from the medical device because it is separable from the medical device or they are both fixed removably.

According to one possible embodiment, another programming method may comprise the following steps:
providing a portable medical device designed to be fixed/attached to the body of the patient, the medical device comprising:
   a casing,
   a reservoir containing the medical fluid to be administered to the patient,
   a processor,
   a pumping mechanism controlled by the processor, designed to administer the fluid contained in the reservoir to the patient,
   a sensor arranged in the casing and designed to send the processor a signal when the casing is removed from and/or fitted to the body of the patient,
   a button designed to send a signal to the processor, and
   a memory coupled to the processor,
activating the sensor a first time so that the medical device initiates an acquisition mode allowing the programming of a given quantity of medical fluid to be administered,
activating the button at least once so as to program the given quantity,
recording the given quantity in the memory,
optionally activating the sensor again so as to send the processor the signal to deliver the fluid in the given quantity recorded in the memory.

The button may be arranged on the casing or on a separate casing. The casing may also contain all or some of the other elements of the medical device (the reservoir, processor, pumping mechanism, memory, etc.).

Optional Additional Security Features

According to one embodiment, before programming the quantity of fluid to be delivered, the user has to apply a pressure (for example a long pressure) to a bolus button of the delivery device so as to confirm his intention to program a bolus. If this long pressure is not performed in a certain lapse of time after the first activation of the sensor or after the further activation of the sensor, the acquisition mode may be canceled.

In order to avoid a build up of bolus programmed via the bolus buttons, if a previous bolus had been programmed via the remote control or via a bolus button and suspended (for example if the patient disconnected his delivery device during delivery of the bolus) then this suspended bolus is canceled in favor of the new programming of a bolus. However, the quantity of fluid already injected may be kept in memory.

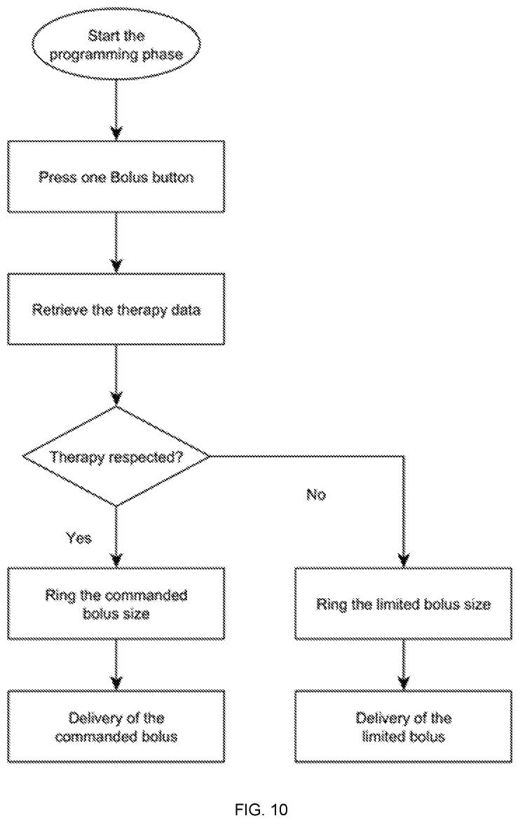

In order to avoid an overdelivery of drug (for example insulin), the delivery device may comprise a memory in which data are recorded regarding the therapy, for example: the maximum quantity of solution that can be administered per bolus, the maximum daily quantity that can be administered and/or at least one previous delivery (quantity and time of execution). When programming a bolus via the bolus buttons, the processor may search the memory for at least one of these data items to ensure that the patient cannot program an inadmissible quantity. FIG. 10 illustrates part of the programming method that takes into consideration at least one data item pertaining to the patient's therapy.

The processor may also estimate and/or take into consideration the quantity of drug already injected but not yet used by the patient's body. This, in the case of insulin, may for example be the IOB (insulin on board). Thus, the processor may verify, for example when activating the acquisition mode, that the IOB is strictly lower than the quantity of insulin that the patient is able to receive. If the IOB exceeds this value then the delivery device will refuse the programming of a new bolus and will indicate this to the patient (vibration and/or ringing and/or red LED).

If the patient attempts to program a quantity that cannot be administered then the delivery device may inform the patient that this dose cannot be delivered followed by the maximum quantity that the device can deliver to the patient. For example, the delivery device may emit a long beep followed by five short beeps in order to inform that the maximum quantity will be 2.5 U even though the patient had programmed a 3 U bolus. The delivery device may be unable to deliver because of an insufficient quantity of fluid in the reservoir or because the therapy does not allow a certain quantity to be exceeded (as divulged hereinabove).

According to one embodiment, the system comprises a remote control designed to exchange data with the delivery device via a two-way wireless communication. This remote control may comprise a large screen with numerous displays as divulged hereinabove. The remote control is to be favored for commanding a bolus because it allows better and more secure programming. This remote control may also prevent the acquisition mode from being activated when the remote control is located within a certain perimeter in which the remote control and the delivery device can exchange data. This embodiment then allows the patient to be encouraged to use the remote control to command a delivery because the remote control is more intuitive and offers a higher level of security than the simple use of buttons arranged on the delivery device. Thus, the patient will be able to use the button or buttons only when his remote control is outside of a certain range, thus rendering the buttons inactive (at least for the programming of a bolus) as long as the remote control is located nearby (which may notably be the case at night, with the remote control being placed on the night stand, thus neutralizing the risk of involuntary manipulation of the buttons as the patient moves around in bed). This given perimeter may be programmed via the remote control or by the doctor and estimated by the communication module which, depending on the strength of the received signal, may estimate the distance between the remote control and the delivery device.

To sum up, if the user were to attempt to command a bolus using the control buttons (as divulged hereinabove) when he could have done so using the remote control, the medical device may refuse the command and/or activate an indication device (a visual, audible or tactile device) and/or the remote control may trigger a specific alert (visual, audible or tactile) to inform the patient that he needs to do the programming using his remote control. Thus, the system may propose, encourage or even require the patient to prioritize issuing his bolus commands using the remote control.

In another embodiment, the delivery device may accept the programming but encourage the patient to validate the command using the remote control. For example, the patient follows the method for programming a bolus as divulged hereinabove but then before initiating the pumping, the delivery device sends the remote control the data set via the bolus buttons and displays these data on the screen of the remote control. The remote control may ring or vibrate as if it had just received a message. Thus, the patient has to use the remote control to validate the set data.

In one embodiment, the bolus buttons can be used to program a bolus even if the remote control could do so. Thus, the patient will be able at his full discretion to program a bolus simply by using the bolus button or buttons. The ringer may be deactivated so that only the tactile indicator is activated by the delivery device (vibration). For example, the patient may be able to program his bolus during a meal without having to get out his remote control and without the other individuals at the table being able to hear the programming operation. However, to avoid overdosing, the delivery device may be set to limit the quantity that can be programmed via the bolus buttons, for example when the remote control can be used to program a bolus. Thus, the delivery device may be set up by the doctor or via the remote control (in which case the system would have a specific display for programming this parameter) to limit the use of the bolus buttons to bolus doses of a maximum of 5 U. This parameter may be recorded in the memory of the delivery device. If the patient attempts to program a 6 U bolus using the bolus buttons then the remote control will demand confirmation of the quantity as divulged hereinabove.

This embodiment is particularly important when the delivery device has no screen designed for viewing the command.

If the command and the confirmation of the command are performed without the remote control, for example because the remote control was out of range at the time of programming, then as soon as the remote control and the medical device are able to exchange data (for example data regarding the previous command using the bolus button or buttons), the remote control may on its screen display an item of information relating to the previous command (for example the quantity delivered (301 in FIG. 7a)) or, if the administration is not finished, the remote control may display the administration in progress with a progress indicator (302 in FIGS. 7b and c). In the latter instance, the remote control can be used to cancel or suspend the delivery.

Other Possible Functionalities of the Buttons of the Delivery Device

Outside of the acquisition mode, the bolus buttons may be inactive, for example as long as the delivery device is coupled or connected to a patch, the control button or buttons could be non-active or, at the very least, actuating them could have no effect.

However, as an option, actuation of this or these buttons outside of acquisition mode may activate a different functionality (than the one set out hereinabove), such as:
suspending all or part of the delivery,
making the remote control ring so that the patient can locate his remote control for example if he has misplaced it,
obtaining information from the delivery device, for example the quantity of the last delivery or the one in progress or some other information recorded in the delivery device (IOB, level in the reservoir, etc.).

Suspending All or Part of the Delivery Using the Button or Buttons

As divulged hereinabove, if the patient wishes to cancel or suspend the current bolus then the patient can reactivate the sensor in order to stop the bolus, for example by disconnecting the delivery device from its patch. In one embodiment, the patient can also temporarily suspend all or part of a delivery (basal and/or bolus) by using the control buttons of the delivery device (also referred to hereinabove as the bolus button); for example, if the patient has forgotten his remote control and is practicing a sport.

With the devices of the prior art, the patient cannot temporarily suspend his basal only by using his remote control. On some devices, he can remove the delivery device (for example: fluidically disconnect the delivery device from its transcutaneous device) to suspend his basal and his bolus but he has to find a safe place in which to temporarily store his delivery device during this time.

Thanks to the control buttons of the delivery device, the patient can also temporarily suspend his basal without having to remove/disconnect his delivery device. For example, the patient can apply a long pressure to one or two bolus buttons to suspend his basal or his bolus without his remote control. A sequence of pressures may also be programmed for activating this functionality. The method for activating this functionality may comprise the following steps:
actuating one or more control buttons in such a way that the button or buttons send the processor a signal ordering the temporary suspension of the delivery of the basal and/or of the bolus,
suspending the delivery for a determined period of time,
after this period of time has elapsed, automatically resuming delivery of the basal and/or of the bolus that has been suspended.

During and/or after the setting of the suspension, the audible or tactile or visual indicator may indicate to the patient that the order has been correctly received.

The period of time may be preprogrammed and/or recorded in a memory of the delivery device. In another instance, this period of time may be set using the control buttons of the delivery device. Thus, even without the remote control or simply discretely, the patient can suspend any delivery or just one type of delivery (basal or bolus) then resume it automatically.

In another scenario, the suspension may be without a time limit. In that case, the delivery device will need to signal repeatedly (every minute or every 20 seconds) that the suspension function has been activated. The method for activating this functionality without a limit on time may comprise the following steps:
actuating one or more control buttons so that the button or buttons send the processor a signal ordering suspension of the delivery of the basal and/or of the bolus;
suspending the delivery;
activating the audible, visual and/or tactile indicator at regular intervals of time for as long as the suspended mode is activated.

In one embodiment, the patient may designate the type of delivery he wishes to suspend, for example using a long pressure for the basal and two short pressures for the bolus.

If there are at least two buttons, the patient may actuate these buttons according to a sequence or actuate them substantially at the same time. This actuation of the buttons may be checked by a security mechanism, for example a computer program contained in the processor so as to avoid the suspension function being activated inadvertently. The sequence need not be overly complex because the consequences of underdelivery (for example of insulin) are not as serious as those of overdelivery.

Making the Remote Control Ring

In one embodiment which may be compatible with the embodiments set out hereinabove, the button or buttons may also be used to find the remote control. Specifically, if the device is close to the remote control and/or if the acquisition mode is not activated, then the use of the buttons of the delivery device may make it possible to make the remote control ring so that the user who has misplaced his remote control can easily locate it. Advantageously, the remote control and/or the delivery device may comprise audible, visual and/or tactile indicators which are activated when the delivery device is distanced from its remote control, thus reminding the user that he is moving away from the remote control or that he has forgotten it.

The patient may thus apply a sequence of pressures or a simultaneous pressure on one or more buttons in order to cause his remote control to ring and/or to vibrate for example until the patient finds it. A specific display on the screen of the remote control may then invite the patient to stop the audible and/or tactile indicator.

Making the Delivery Device Ring

In one embodiment which may be compatible with the embodiments divulged hereinabove, the bolus buttons may be actuated by the user in such a way as to interrogate the delivery device, for example in order to determine:
the data item or items relating to the previous delivery (time of execution and/or quantity delivered), and/or
the data item or items relating to the delivery in progress (programmed quantity, delivered or yet to be delivered, of the current bolus, flow rate of the basal, etc.), and/or
the data item or items relating to the quantity left in the reservoir; and/or
the data item or items relating to the quantity of medical fluid injected into the body of the patient but not yet used (IOB, etc.), etc.

The patient may perform a specific actuating sequence of the bolus button or buttons in order to find the data item or items desired.

For example, if the patient applies a long pressure followed by a short pressure in order to determine the quantity delivered during the previous bolus, the delivery device will activate its audible, tactile and/or visual indicator in pulses in order to inform the patient, it being possible for 6 pulses to signal 3 U delivered during the previous bolus. The time of the delivery may also be given for example in the manner of the minute chimes of mechanical watches (the sound is different according to the hour, quarter hour and minutes). The delivery device may also be provided with a loudspeaker so that the information is given and heard intelligibly by the patient (for example an automated voice).

Likewise, information about the IOB may be important to the patient, and two successive long pressures may allow the processor to be sent a signal so that the device will estimate the current IOB and inform the patient of it, for example as divulged hereinabove.

The actuating sequences for the bolus button or buttons may be different and customizable.

The sequence or sequences may be customizable for example using the remote control. Thus, a specific display may allow such customization of the sequences.

Furthermore, in order to limit the complexity of use, the patient may choose (for example via a programming screen of a computer or of the remote control) which functionality or functionalities will be accessible via the control button or buttons of the delivery device. The programming screen (as divulged in FIG. 16) may be adapted to list one or more functionalities, each of these functionalities being able to be activated or deactivated by the patient or by the doctor. Thus, according to these customization data, the bolus button or buttons will be able to provide access to only a certain functionality, for example the bolus command and the reservoir level or information relating to the previous bolus or to the IOB, etc. The programming screen may for each of these functionalities comprise a button which activates or deactivates the functionality. Another screen may allow the selection of the button actuating sequence, and the selection of the button or buttons used for each of these functionalities. These customization data may then be sent to the delivery device so that it can be customized.

In this way, the function and functions associated with the control button of the delivery device can be customized, something which is particularly important when the patient is a child. Thus, the doctor or the parents (or a responsible individual) can customize the delivery device to render operative or inoperative all or some of the functionalities set out in the present document. For example, the doctor may elect to deactivate the ability to command the bolus using the bolus buttons, so as to protect the patient.

Thus, all of these embodiments may be compatible with one another and allow the patient to improve his experience of use with or without remote control.

1-26. (canceled)

27: A method for commanding an administration of a medical fluid to a patient, the method comprising the steps of:
providing a portable medical device configured to be attached to a body of the patient, the portable medical device comprising, a reservoir holding the medical fluid to be administered to the patient, a processor, a pumping mechanism controlled by the processor, configured to administer the fluid held in the reservoir to the patient, a button configured to send a signal to the processor related to the administration of the medical fluid upon activating the button, a communication device, and a memory connected to the processor;
providing a remote control for the portable medical device configured to communicate with the portable medical device via the communication device;
determining whether at least one of (i) the medical device and the remote control can communicate with each other, and (ii) the medical device and the remote control are within a certain perimeter from each other; and
deactivating the button for a command regarding the administration of the medical fluid, after the step of determining has determined that at least one of (i) the medical device and the remote control can communicate with each other, and (ii) the medical device and the remote control are within a certain perimeter from each other.

28: The method as claimed in claim 27, further comprising the step of:
providing an alert when the button is activated, after the step of determining has determined that at least one of (i) the medical device and the remote control can communicate with each other, and (ii) the medical device and the remote control are within a certain perimeter from each other.

29: The method as claimed in claim 27, further comprising the step of:
activating the button for providing the command regarding the administration of the medical fluid, after the step of determining has determined that at least one of (i) the medical device and the remote control cannot communicate with each other, and (ii) the medical device and the remote control are not within a certain perimeter from each other.

30: The method as claimed in claim 29, further comprising the step of:
entering data related to the command regarding administration of the medical fluid with the remote control;
sending the data to the portable medical device; and
storing the data in the memory of the portable medical device.

31: The method as claimed in claim 29, further comprising the step of:
entering data related to the command regarding administration of the medical fluid with the button, and
storing the data in the memory of the portable medical device.

32: The method as claimed in claim 29, further comprising the step of:
cancelling the command by the portable medical device if the button is not actuated after a determined period of time following the step of activating.

33: The method as claimed in claim 27, further comprising the step of:
providing a signal by the remote control to encourage use of the remote control, after the step of determining.

34: The method as claimed in claim 33, wherein in the step of providing the signal, a ringing or a vibrating signal is provided.

35: The method as claimed in claim 30, further comprising the step of:
deactivating a generation of a command regarding the administration of the medical fluid after detecting a removal of the portable medical device from a patch associated with the portable medical device.

36: A system for commanding an administration of a medical fluid to a patient, the system comprising:
a portable medical device configured to be attached to a body of the patient, the portable medical device including, a reservoir holding the medical fluid to be administered to the patient, a processor, a pumping mechanism controlled by the processor, the pumping mechanism configured to administer the fluid held in the reservoir to the patient, a button configured to send a signal to the processor related to the administration of the medical fluid upon activating the button, a communication device, and a memory connected to the processor; and
a remote control for the portable medical device having a processor, the remote control configured to communicate with the portable medical device via the communication device,
wherein the processor of the portable medical device is configured to
determine whether at least one of (i) the medical device and the remote control can communicate with each other, and (ii) the medical device and the remote control are within a certain perimeter from each other,
deactivate the button for a command regarding the administration of the medical fluid, after the determining that at least one of (i) the medical device and the remote control can communicate with each other, and (ii) the medical device and the remote control are within a certain perimeter from each other.

37: The system as claimed in claim 36, wherein the processor of the portable medical device is configured to
provide an alert when the button is being activated, after determining that at least one of (i) the medical device and the remote control can communicate with each other, and (ii) the medical device and the remote control are within a certain perimeter from each other.

38: The system as claimed in claim 36, wherein the processor of the portable medical device is configured to
activate the button for providing the command regarding the administration of the medical fluid, after the step of determining has determined that at least one of (i) the medical device and the remote control cannot communicate with each other, and (ii) the medical device and the remote control are not within a certain perimeter from each other.

39: The system as claimed in claim 38, wherein the processor of the portable medical device is configured to
receive data related to the command regarding administration of the medical fluid from the remote control, and
store the data in the memory of the portable medical device.

40: The system as claimed in claim 38, wherein the processor of the portable medical device is configured to
receive data related to the command regarding administration of the medical fluid with an activation of the button, and
store the data in the memory of the portable medical device.

41: The system as claimed in claim 38, wherein the processor of the portable medical device is configured to
cancel the command by the portable medical device if the button is not actuated after a determined period of time following the activating.

42: The system as claimed in claim 36, wherein the processor of the remote control is configured to
cause a generation of a signal by the remote control to encourage use of the remote control, after the determining.

43: The system as claimed in claim 42, wherein in the generation of the signal, a ringing or a vibrating signal is provided.

44: The system as claimed in claim 38, wherein the processor of the portable medical device is configured to
deactivate a generation of a command regarding the administration of the medical fluid after detecting a removal of the portable medical device from a patch associated with the portable medical device.

45: A system for commanding an administration of a medical fluid to a patient, the system comprising:
a portable medical device configured to be attached to a body of the patient, the portable medical device including, a reservoir holding the medical fluid to be administered to the patient, a processor, a pumping mechanism controlled by the processor, the pumping mechanism configured to administer the fluid held in the reservoir to the patient, a button configured to send a signal to the processor related to the administration of the medical fluid upon activating the button, a communication device, and a memory connected to the processor; and
a remote control for the portable medical device having a processor, the remote control configured to communicate with the portable medical device via the communication device,
wherein the processor of the portable medical device is configured to
determine whether at least one of (i) the medical device and the remote control can communicate with each other, and (ii) the medical device and the remote control are within a certain perimeter from each other,
activate the button for providing a command regarding the administration of the medical fluid, after the determining that at least one of (i) the medical device and the remote control cannot communicate with each other, and (ii) the medical device and the remote control are not within a certain perimeter from each other.

46: The system as claimed in claim 45, wherein the processor of the portable medical device is configured to
deactivate the button for providing the command regarding the administration of the medical fluid, after the step of determining has determined that at least one of (i) the medical device and the remote control can communicate with each other, and (ii) the medical device and the remote control are within a certain perimeter from each other.

47: The system as claimed in claim 45, wherein the processor of the portable medical device is configured to
cancel the command by the portable medical device if the button is not actuated after a determined period of time following the activating.

48: The system as claimed in claim 45, wherein the processor of the portable medical device is configured to
deactivate the button for providing the command regarding the administration of the medical fluid after detecting a removal of the portable medical device from a patch associated with the portable medical device.
